(19) United States
(12) Patent Application Publication
(10) Pub. No.: US 2004/0096494 A1
(43) Pub. Date: May 20, 2004

(54) Composition

(76) Inventors: Britta Siekmann, Lund (SE); Barbo Thoring, Sodertalje (SE)

Correspondence Address: WHITE & CASE LLP, PATENT DEPARTMENT, 1155 AVENUE OF THE AMERICAS, NEW YORK, NY, 10036, US

(21) Appl. No.: 10/471,378

(22) PCT Filed: Mar. 13, 2002
(86) PCT No.: PCT/SE02/00476

(51) Int. Cl. A61K 31/381; A61K 31/21; A61K 9/127

(52) U.S. Cl. 424/450; 514/438; 514/509

(57) ABSTRACT

A new pharmaceutical composition in the form of lipoglobules which comprises (a) one or more NO-releasing NSAID(s); (b) one or more surfactant(s); and (c) an aqueous phase, as well as a process for the preparation of such composition and the use of such composition in the treatment of pain and inflammation.

### FIELD OF THE INVENTION

The present invention is related to a new pharmaceutical composition in the form of lipoglobules which comprises (a) one or more NO-releasing NSAID; (b) one or more surface active agent(s); and (c) an aqueous phase, and to a process for the preparation of such composition. The claimed composition is intended for oral, topical, rectal, nasal and parenteral administration in humans and animals. The present invention also relates to the use of the new composition in the treatment of pain and inflammation.

### BACKGROUND AND PRIOR ART

Nitrogen oxide releasing nonsteroidal antiinflammatory drugs (in the following named NO-releasing NSAIDs or shorter NO-NSAIDs) have recently been found to have an improved side-effect profile, see e.g. WO 94/04484, WO 94/12463, WO 95/09831 and WO95/30641, compared to the well-known drugs used in the treatment of pain and inflammation, NSAIDs. Patients undergoing treatment with NSAIDs for a longer period of time often experience problems with stomach gastrointestinal side-effects.

NO-NSAIDs are in general lipophilic compounds with poor aqueous solubility. NO-NSAIDs are practically insoluble in water. This inherent property of NO-NSAIDs poses a number of problems to the formulator. Upon oral administration, the absorption of NO-NSAIDs from the gastrointestinal tract (GIT) may be dissolution rate limited due to poor solubility in gastrointestinal fluids, which in turn results in poor bioavailibility. For parenteral, in particular intravenous administration, an aqueous based formulation is required which provides sufficient solubility of the NO-NSAID compound to reach therapeutic plasma levels.

Surfactants are known to be able to increase the solubility of poorly water soluble compounds. Different types of surfactant based drug delivery systems are known, such as micellar solutions, vesicular systems, e.g. liposomes, and emulsions.

Micellar solutions comprise the drug solubilised in a surfactant aggregate, e.g. spherical micelles, in an aqueous medium. Typically, the diameter of these aggregates is in the order of two molecular lengths of.the surfactant molecule, i.e. some ten to hundred Ångström. According to the Gibbs phase law, micellar solutions represent one phase systems. Disadvantages of micellar systems are that the solubility enhancement by the surfactant is usually only modest, or that high surfactant-to-drug ratios are required to obtain sufficient solubility. A high surfactant load is not desirable from a toxicological point of view. Upon administration of micellar systems, there is a risk that the drug may precipitate when the micellar system is diluted in gastrointestinal fluids or in the blood. In oral administration, precipitation may lead to reduced bioavailability. In intravenous administration, drug precipitation may lead to pain upon injection, venal tissue irritation, and embolism.

Vesicles are bilayer systems in which an aqueous space is surrounded by one (unilamellar) or more (oligo- and multilamellar) surfactant bilayers. In liposomes these bilayers consist of phospholipids. Hydrophilic drugs can be incorporated in the internal aqueous phase whereas lipophilic drugs partition into the surfactant bilayer. Vesicle dispersions are two phase systems. Typically, the vesicle diameter is in the nanometer to micrometer range depending on the number of bilayers. The amount of lipophilic drug that can be incorporated into the surfactant bilayers is usually low because the drug may disturb the bilayer structure leading to instability.

Emulsions represent dispersions of one liquid in another, not miscible liquid, typically by the aid of a surfactant acting as an emulsifier. Two basic types can be distinguished, oil-in-water (o/w) and water-in-oil (w/o). Oil-in-water emulsions comprise an aqueous continuous phase in which oil droplets are dispersed. In w/o emulsions an aqueous phase is dispersed in an oily continuous medium. For intravenous administration, only the o/w emulsions can be used, provided that the size of the oil droplets is small enough to prevent blockage of blood capillaries. Submicron sized o/w emulsions have been used in parenteral nutrition for a long time. Emulsions as delivery systems for poorly water soluble drugs comprise at least four components, (a) a drug, (b) a lipid phase, (c) an emulsifier, and (d) an aqueous phase. The poorly water soluble drug is usually dissolved in the lipid phase. Thus, in this case the lipid phase is used to solubilise the drug whereas the surfactant serves as a dispersion aid and as a stabilisor of the oil phase. As with micellar and vesicular systems, the solubilisation capacity of o/w emulsions is generally low. It is determined by the solubility of the drug in the oil phase.

Outline of the Invention

It has now surprisingly been found that the problems outlined above can be solved by a novel type of surfactant based delivery system for NO-NSAIDs, a pharmaceutical composition in the form of lipoglobules.

The present invention discloses pharmaceutical compositions in the form of lipoglobules comprising the following components

(a) one or more NO-releasing NSAID(s);

(b) one or more surfactant(s); and

(c) an aqueous phase

wherein the NO-releasing NSAID(s) is a lipophilic core surrounded by one or more layers of surfactant(s), which NO-releasing NSAID(s) and surfactant(s) are dispersed in an aqueous phase.

Optionally the NO-NSAID compound(s) can be mixed with one or more lipophilic water-immiscible solvent(s), e.g. in order to adjust the density difference between the aqueous and the oil phase. The density of NO-NSAIDs is usually greater than that of water, and adjustment of densities may be advantageous to prevent sedimentation of the NO-NSAID lipoglobules. Density adjustment can also be obtained by increasing the density of the aqueous phase, e.g. by adding sugars, sugar alcohols or salt.

Depending on solubility, the surfactant(s) can be dissolved in either the aqueous or the lipophilic phase. One of the unique features with NO-NSAIDs is that many of these lipophilic compounds are oils or thermosoftening semisolids which are practically insoluble in water. They can thus serve as the oil phase as such, of an o/w emulsion. These compounds can be emulsified in an aqueous phase by a surfactant providing lipoglobules consisting of the NO-NSAID compound(s) as a core surrounded by one or more surfactant monolayers and dispersed in an aqueous medium. The surfactant layer stabilises the lipoglobules against aggregation and coalescence. Thermbsoftening NO-NSAIDs may be heated above their melting point prior to emulsification to facilitate homogenisation, or may be dissolved in a liquid NO-NSAID or in another lipophilic, water-immiscible solvent.

Preferred NO-releasing NSAIDs in accordance with the present invention, are compounds of the formula I

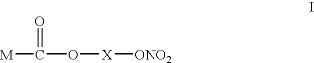

wherein

X is a spacer, i.e. a compound forming a bridge between the nitrogen oxide donating group and the NSAID; and

M is selected from anyone of

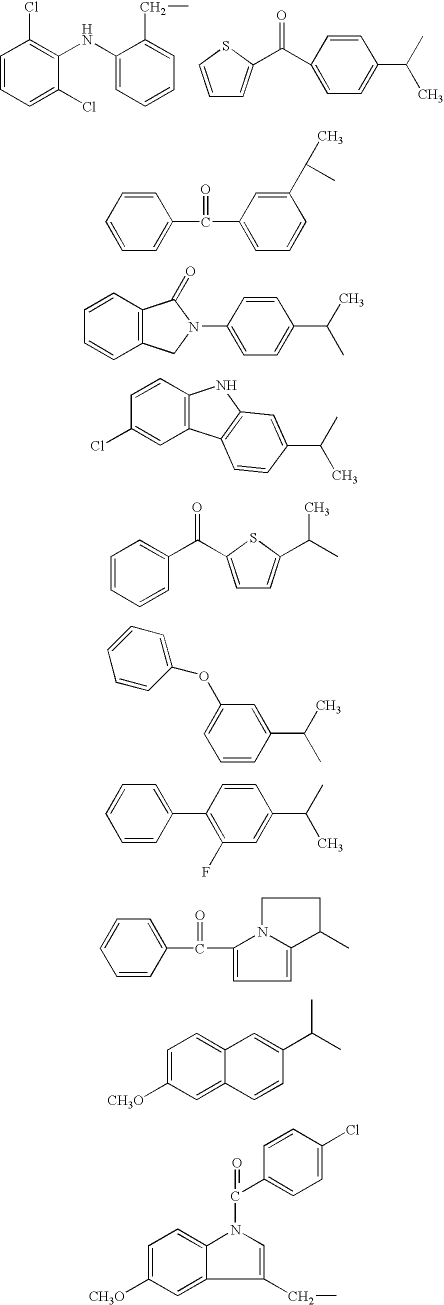

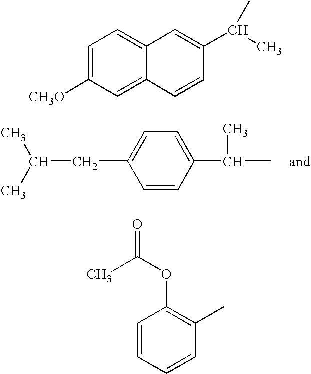

In a preferred embodiment of the invention, the spacer X is selected from a linear, branched or cyclic alkylene group —(CH₂)—ₙ wherein n is an integer of from 2 to 10; and —(CH₂)ₘ—O—(CH₂)ₚ— wherein m and p are integers of from 2 to 10; and —CH₂—pC₆H₄—CH₂—.

In one embodiment of the invention, NO-NSAIDs contemplated as active compounds in the compositions according to the present invention, are compounds disclosed and claimed in WO 94/04484, WO 94/12463, WO 95/09831 and WO 95/30641, which are hereby incorporated by reference.

Specific NO-releasing substances useful in accordance with the present invention are

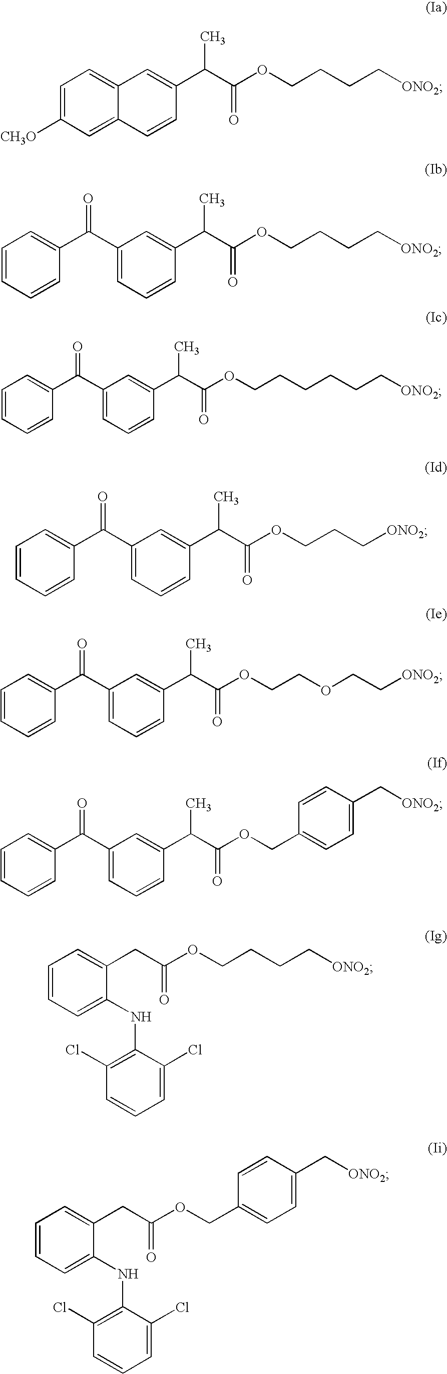

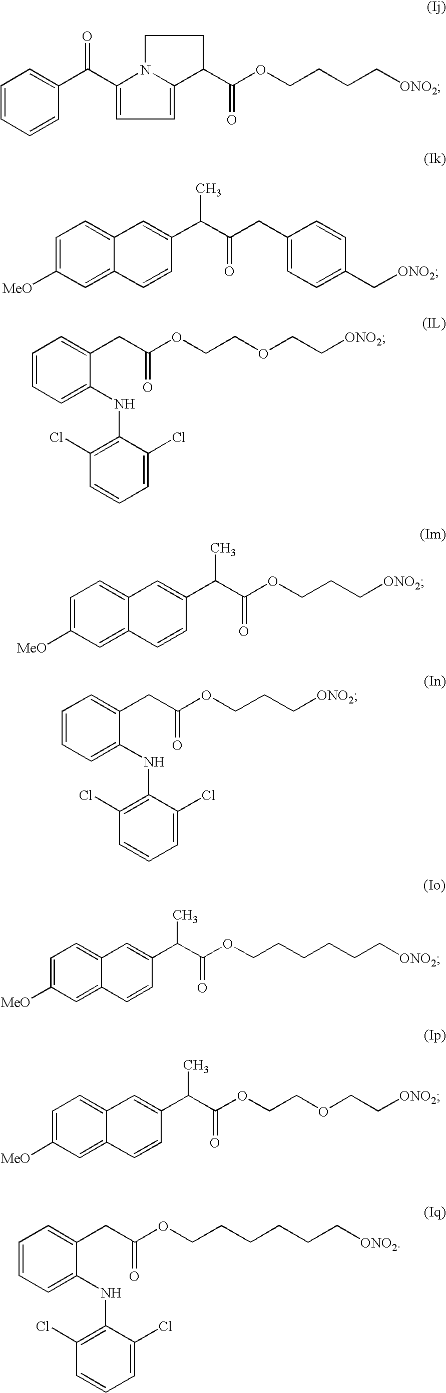

Most preferred NO-NSAIDs useful according to the invention are compounds of formulas Ia and Ig.

Suitable surfactants include, but are not limited to, phospholipids, e.g. naturally occurring phospholipids such as egg and soy lecithin; synthetic or semisynthetic phospholipids such as phosphatidylcholines, phosphatidylethanolamines, phosphatidylglycerols, phosphatidylinositols and phosphatidic acids; ethoxylated phospholipids such as polyoxyethylen-phosphatidylethanolamine; galactolipids and other glycolipids; bile acids such as cholic acid, taurocholic acid and glycocholic acid and their salts; sterols such as cholesterol, sitosterol, sitostanol and esters therof; ethoxylated sterols such as polyoxyethylene sitosterol; fatty acids and their salts; mono- and diglyceride esters of fatty acids, e.g. monooleate and monostearate; fatty acid esters and alcohols; ethoxylated fatty acids, ethers and esters; ethoxylated castor oil, e.g. Cremophor EL; ethoxylated sorbitan esters such as polysorbates, e.g. polysorbate 80 (Tween 80); polypropylene-polyethylene block copolymers such as poloxamers, e.g. Poloxamer 188 and Poloxamer 407, and poloxamines, e.g. Tetronic 908; or a mixture of two or more of these surfactants.

Preferably the surfactant is one of a naturally occuring, synthetic or semi-synthetic phospholipid; a polypropylene polyethylene block copolymer; an ethoxylated sorbitan ester; or a mixture of two or more of these surfactants.

More preferred the surfactants is a naturally occuring phospolipid from soya in combination with a poloxamer, preferably poloxamer 407; or polysorbate 80.

A wide range of lipophilic, water-immiscible solvents can be used in the compositions of the present invention. Typically the water-immiscible solvent is a vegetable oil, e.g. soy bean, arachis, castor, corn, cottonseed, olive, safflower or sunflower oil. Suitable solvents also include fractionated oils such as fractionated coconut oil. The water-immiscible solvent may also be a marine oil such as cod liver oil or other fish oils, also known as omega-3 polyunsaturated oils. Alternatively, the water-immiscible solvent is an ester of a medium or long-chain fatty acid, for example a mono-, di-, or triglyceride; or is a chemically modified or manufactured material such as ethyl oleate, isopropyl myristate, isopropyl palmitate, a glycerol ester or polyoxyl hydrogenated castor oil. The compositions of the present invention may comprise a mixture of NO-NSAID and one or more of the above water-immiscible solvents.

The aqueous phase comprises water and may—depending on the intended way of administration—optionally contain buffering agents and salts; pH adjusting agents such as sodium hydroxide and hydrochloric acid; tonicity modifiers such as glycerol, xylitol, sorbitol, mannitol, and glucose; water-miscible solvents such as glycerol, ethanol, polyethylene glycol and propylene glycol; density modifiers such as polyols, sugars, sugar alcohols and salts; viscosity modifiers such as thickeners and gelling agents; preservatives such as chlorhexidine, methyl-, ethyl-, propyl- or butylparaben, and thimerosal; antioxidants such as ascorbic acid and tocopherol derivates; taste modifiers such as sugars, sweeteners and flavouring agents.

A composition of the present invention typically comprises one or more NO-NSAID(s) or mixtures of one or more NO-NSAID(s) and one ore more water-immiscible solvent(s) in an amount that is up to 30% by weight of the composition, preferably 0.5-20%. The surfactant or surfactant mixture may be present in an amount up to 20% by weight of the composition, preferably 0.1-10%.

The dispersion techniques used in preparation of the present lipoglobule formulations can be conventional dispersion techniques such as high shear stirring, ultraturrax vortexing, sonication, high pressure homogenisation and microfluidisation. Preferably high pressure homogenisation or microfluidisation are used. The globule size is a function of the composition and dispersion parameters. As a general rule, globule size decreases with increasing amount of surfactant or with decreasing amount of the oil phase. Globule size also decreases with increasing energy input during dispersion until it levels off. Further energy input may lead to an increase in globule size, an effect known as overemulsification.

The globule size of the present lipoglobules is typically in the nanometer and micrometer range, more specifically from 50 nm to 50 μm, preferably 200 nm to 5 μm. Control of globule size is of importance for parenteral, in particular intravenous formulations. For intravenous administration, the average globule size should be below 1 μm, preferably 200-500 nm, with basically no globules above 5 μm present.

The pharmaceutical compositions in form of lipoglobules according to the present invention are suitable for oral, parenteral, topical, nasal and rectal administration of NO-NSAIDs. Where a formulation is to be used for parenteral administration, it must be sterile. Sterilisation is preferably performed by autoclavation. Ingredients in formulations for parenteral administration will have to be of injection grade and approved for such administration. Topical formulations should preferably be viscous and spreadable unless they are included in a patch.

The total amount of NO-NSAIDs used in the compositions of the invention is preferably in the range of 50-1500 mg per unit dose. In still a further preferred embodiment the amount of NO-NSAIDs used in the composition is 125-500 mg per unit dose.

The pharmaceutical lipoglobule composition of the present invention is particularly useful in the treatment of pain and inflammation. The wording “pain” is intended to include, but not limited to, nociceptive and neuropathic pain or combinations thereof; acute, intermittent and chronic pain; cancer pain; migraine and headaches of similar origin. The wording “inflammation” is intended to include, but not limited to, rheumatoid arthritis; ostheoarthritis; and juvenile arthritis.

Methods of Preparation

The compositions according to present invention may be prepared according to one of the is following processes wherein

i) one or more surfactant(s) is added to the aqueous phase whereupon one or more NO-NSAID(s) is dispersed in the aqueous phase by using conventional dispersion techniques such as high shear mixing, sonication or high pressure homogenisation; or

ii) one or more NO-NSAID(s) is mixed with one or more surfactant(s), whereupon the mixture is dispersed in the aqueous phase by using conventional dispersion techniques such as high shear mixing, sonication or high pressure homogenisation; or

iii) one or more surfactant(s) is added to the aqueous phase and one or more NO-NSAID(s) is mixed with one or more lipophilic water-immiscible solvent(s), whereupon the mixture of NO-NSAID(s) and lipophilic immiscible solvent(s) is dispersed in the aqueous phase by using conventional dispersion techniques such as high shear mixing, sonication or high pressure homogenisation;

iv) one or more NO-NSAID(s) is mixed with one or more surfactant(s) and one or more lipophilic water-immiscible solvent(s), whereupon the mixture is dispersed in the aqueous phase phase by using conventional dispersion techniques such as high shear mixing, sonication or high pressure homogenisation;

v) one or more surfactant(s) is added to the aqueous phase, and one or more surfactant(s) is mixed with one or more NO-NSAID(s), whereupon the mixture of NO-NSAID(s) and surfactant(s) is dispersed in the aqueous phase by using conventional dispersion techniques such as high shear mixing, sonication or high pressure homogenisation; or

vi) one or more surfactant(s) is added to the aqueous phase, and one or more surfactant(s) as well as one or more lipophilic water-immiscible solvent(s) is mixed with one or more NO-NSAID(s) whereupon the mixture of NO-NSAID(s), surfactant(s) and lipophilic water-immiscible solvent(s) is dispersed in the aqueous phase by using conventional dispersion techniques such as high shear mixing, sonication or high pressure homogenisation.

Thermosoftening NO-NSAIDs may be heated above their melting point prior to emulsification to facilitate homogenisation, or may be dissolved in a liquid NO-NSAD or in another lipophilic, water-immiscible solvent.

### DETAILED DESCRIPTION OF THE INVENTION

The invention will now be described in more detail by the following examples, which are not to be construed as limiting the invention.

### EXAMPLE 1

| | Ex. 1.1 | Ex. 1.2 | Ex. 1.3 | Ex. 1.4 | Ex. 1.5 |
|---|---|---|---|---|---|
| Composition | mg/g | mg/g | mg/g | mg/g | mg/g |
| Compound of formula Ia | 0.77 | 1.30 | 1.06 | 1.06 | 21.2 |
| Fractionated coconut oil | 2.97 | 4.90 | 4.00 | 100.1 | 79.9 |
| Phospholipon 80 | 0.76 | 1.32 | 1.08 | 21.6 | 21.6 |
| Poloxamer 407 | 1.61 | 2.81 | 2.30 | 45.9 | 45.9 |
| Water | To 1000 | To 1000 | To 1000 | To 1000 | To 1000 |

Preparation

1. Aqueous phase: Fractionated soya phospholipid (Phospholipon 80) and poloxamer 407 (Lutrol F127) were dispersed in water with an Ultra Sonic rod or a high shear mixer.

2. Oil phase: Compound of formula Ia and coconut oil were mixed by hand stirring during heating to maximum 60° C.

3. The aqueous phase and oil phase were poured together. Emulsion was formed by sonication with an ultra sonic rod, or by first mixing with a high shear mixer and then homogenising with a high pressure homogeniser, until average droplet size is <300 nm (as measured by photon correlation spectroscopy in a Malvern PCS 4700).

Optionally the emulsion was autoclaved (15 min at 121° C.) to prevent microbiological growth, and then stored at room temperature for at least 6 months.

The oral bioavailability of compound of formula Ia in lipoglobules of example 1.1, measured as the relative bioavailability of its metabolite naproxen (analysed naproxen-plasma level relative to given dose of compound of formula Ia), was 88% in rat (4 ml/kg).

### EXAMPLE 2

| | Ex. 2.1 | Ex. 2.2 |
|---|---|---|
| Composition | mg/g | mg/ml |
| Compound of formula Ia | 0.87 | 1.30 |
| Fractionated coconut oil | 3.28 | 4.87 |
| Polysorbate 80 | 1.38 | 2.06 |
| Sodium-Carboxy metyl | 14.6 | 14.9 |
| cellulose, medium viscous | | |
| Water | To 1000 | To 1000 |

Preparation

1. Oil phase: Compound of formula Ia and coconut oil were mixed by hand stirring during heating to maximum 60° C.

2. Polysorbate was added to the oil phase whereafter the mixture was heated to 60° C. and stirred for 1 minute with a high shear mixer.

3. Water heated to 60° C. was added in small portions while stirring with high shear mixer. In total the amount of water was approximately twice the amount of oil phase in step 1.

4. The mixture was stirred with high shear mixer for 2 minutes at 60° C.

5. Stirring with high shear mixer for 2 minutes while cooling to room temperature.

6. Water was added in an amount enough to double the amount of emulsion whereafter the mixture was mixed until homogeneous.

7. Sodium-carboxymethylcellulose suspension, mediumviscous, 1.5% in water was added. Stirring with magnet for 10 minutes.

Mean droplet size is <2 μm, 90% of the droplets are <5 μm (as measured by laser diffraction in a Coulter LS230).

The oral bioavailability of compound of formula Ia in example 2.1, measured as the relative bioavailability of its metabolite naproxen (analysed naproxen-plasma level relative to given dose of compound of formula la), was 95% in rat (4 ml/kg).

### EXAMPLE 3

| | Ex. 3.1 |
|---|---|
| Composition | mg/g |
| Compound of formula Ia | 187.5 |
| Polysorbate 80 | 62.5 |
| Water | 750.0 |

Preparation

1. Oil phase: Compound of formula Ia and Polysorbate were mixed with high shear mixer at temperature maximum 60° C.

2. Water heated to 60° C. was added in small portions while stirring with high shear mixer. In total the amount of water was approximately twice the amount of oil phase in step 1.

3. Stirring with high shear mixer for 2 minutes at 60° C.

4. Stirring with high shear mixer for 2 minutes while cooling to room temperature.

5. The rest of the water was added and mixed with magnet until homogeneous.

Mean droplet size is <2 μm, 90% of the droplets are <5 μm (measured with LS).

### EXAMPLE 4

| | Ex. 4.1 |
|---|---|
| Composition | mg/g |
| Compound of formula Ig | 0.25 |
| Fractionated coconut oil | 0.94 |
| Phospholipon 80 | 0.25 |
| Poloxamer 407 | 0.54 |
| Water | To 1000 |

Preparation

1. Aqueous phase: Fractionated soya phospholipid (Phospholipon 80) and poloxamer 407 (Lutrol F127) were dispersed in water with suitable mixing equipment.

2. Oil phase: Compound of formula Ig and coconut oil were mixed during gentle stirring.

3. The aqueous phase was slowly added to the oil phase during stirring. The emulsion was homogenised, e.g. with an ultra sonic rod or homogeniser, to eliminate the risk of large droplets.

90% or more of the droplets formed have a particle size smaller than 0.2 μm.

### EXAMPLE 5

| | Ex. 5.1 |
|---|---|
| Composition | mg/g |
| Compound of formula Ig | 0.413 |
| Fractionated coconut oil | 99.6 |
| Poloxamer 407 | 19.8 |
| Water | To 1000 |

| | Ex. 5.2 |
|---|---|
| Composition | mg/g |
| Compound of formula IL | 0.429 |
| Fractionated coconut oil | 100 |
| Poloxamer 407 | 19.8 |
| Water | To 1000 |

| | Ex. 5.3 |
|---|---|
| Composition | mg/g |
| Compound of formula Ic | 0.357 |
| Fractionated coconut oil | 99.6 |
| Poloxamer 407 | 19.8 |
| Water | To 1000 |

| | Ex. 5.4 |
|---|---|
| Composition | mg/g |
| Compound of formula If | 0.419 |
| Fractionated coconut oil | 99.6 |
| Poloxamer 407 | 19.8 |
| Water | To 1000 |

Preparation

1. Oil-phase: The NO-releasing compound of formula Ig, IL, Ic and If, respectively, was mixed with the coconut oil by stirring. Heating to max 40° C. was used if needed.

2. Aqueous phase: The poloxamer 407 was dispersed in the water by high-shear mixer.

3. The aqueous phase and the oil phase were mixed together. Emulsion was formed by first mixing with a high shear mixer and then homogenising with a high-pressure homogeniser.

Mean droplet size was 0.13-0.15 μm, 99% of the droplets were <0.23-0.25 μm (as measured by laser diffraction in a Coulter LS230).

The oral bioavailability of compound of formual Ig and compound of formula IL in lipoglobules, expressed as the systemic exposure to diclofenac (their active metabolite) relative to the systemic exposure following intravenous administration of diclofenac, was 85% and 104% respectively in the minipig (5 ml/kg).

The oral bioavailability of compound of formula Ic and compound of formula If in lipoglobules, expressed as the systemic exposure to ketoprofen (their active metabolite) relative to the systemic exposure following intravenous administration of ketoprofen, was 82% and 80% respectively in the minipig (5 ml/kg)

### EXAMPLE 6

| | Ex. 6.1 |
|---|---|
| Composition | mg/g |
| Compound of formula Ia | 20.8 |
| 3H-labelled Compound of formula Ia | 7 × 10⁻⁸ |
| Poloxamer 407 | 4.16 |
| Water | To 1000 |

Preparation

1. Aqueous phase: Poloxamer 407 was dissolved in cold water over night.

2. Oil phase: Compound of formula Ia and 3H-labelled compound of formula Ia dissolved in ethanol were mixed by adding more ethanol. The ethanol was then evaporated.

3. The aqueous phase and the oil phase were mixed together. Emulsion was formed by sonication with an ultra sonic rod.

At in-vitro permeation studies with human skin a steady state flux between 0.20-0.72 μg/cm²/h was achieved.

### EXAMPLE 7

| | Ex. 7.1 |
|---|---|
| Composition | mg/g |
| Compound of formula Ia | 20.8 |
| Fractionated coconut oil | 78.2 |
| Poloxamer 407 | 19.8 |
| Water | To 1000 |

Preparation

1. Oil-phase: The NO-releasing compound was mixed with the coconut oil by stirring. Heating to max 60° C. was used. The poloxamer 407 was dissolved in the oil-mixture during heating to max 60° C.

2. The water and the oil phase was poured together. Emulsion was formed by first mixing with a high shear mixer and then homogenising with a high-pressure homogeniser.

Optionally the emulsion was heat-treated (≦15 min at 121° C.) to prevent microbiological growth.

Mean droplet size was <0.5 μm, 99% of the droplets were <2 μm (as measured by laser diffraction in a Coulter LS230).

1. A pharmaceutical composition in form of lipoglobules comprising
(a) one or more NO-releasing NSAID(s);
(b) one or more surfactant(s); and
(c) an aqueous phase
wherein the NO-releasing NSAD(s) is a lipophilic core surrounded by one or more layers of surfactant(s), which NO-releasing NSAID(s) and surfactant(s) are dispersed in an aqueous phase.

2. A pharmaceutical composition according to claim 1 wherein the NO-releasing NSAID is a compound of the formula I 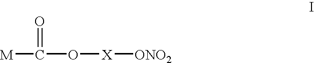
wherein
X is a spacer, and
M is selected from anyone of

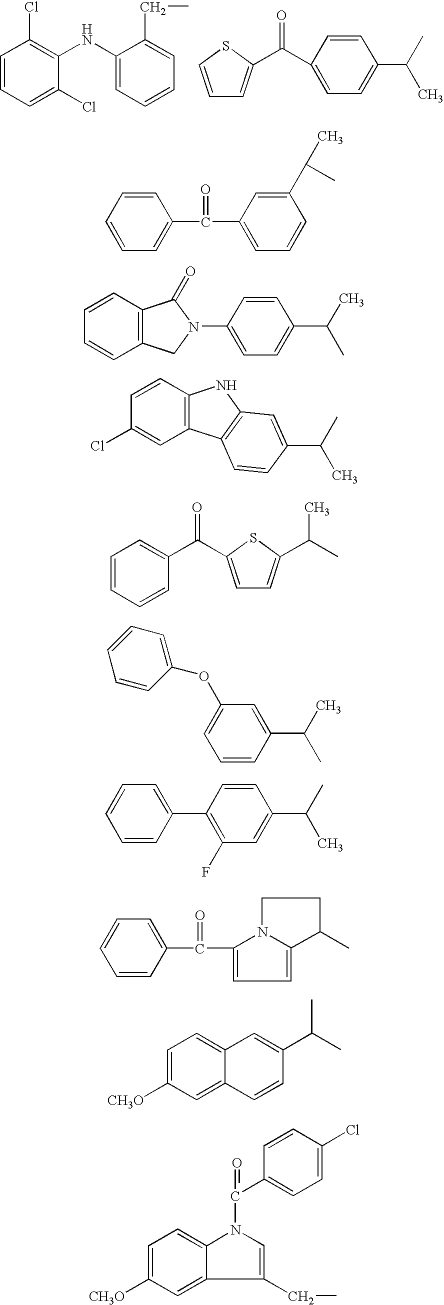

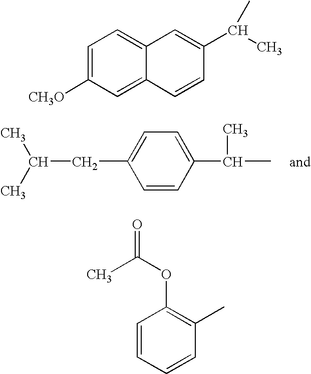

3. A pharmaceutical composition according to claim 2, wherein the spacer X of the NO-releasing NSAID is selected from a linear, branched or cyclic alkylene group —(CH₂)—ₙ wherein n is an integer of from 2 to 10; —(CH₂)ₘ—O—(CH₂)ₚ— wherein m and p are integers of from 2 to 10; and —CH₂—pC₆H₄—CH₂—.

4. A pharmaceutical composition according to any one of the preceding claims, wherein the NO-releasing NSAID is any one compound selected from

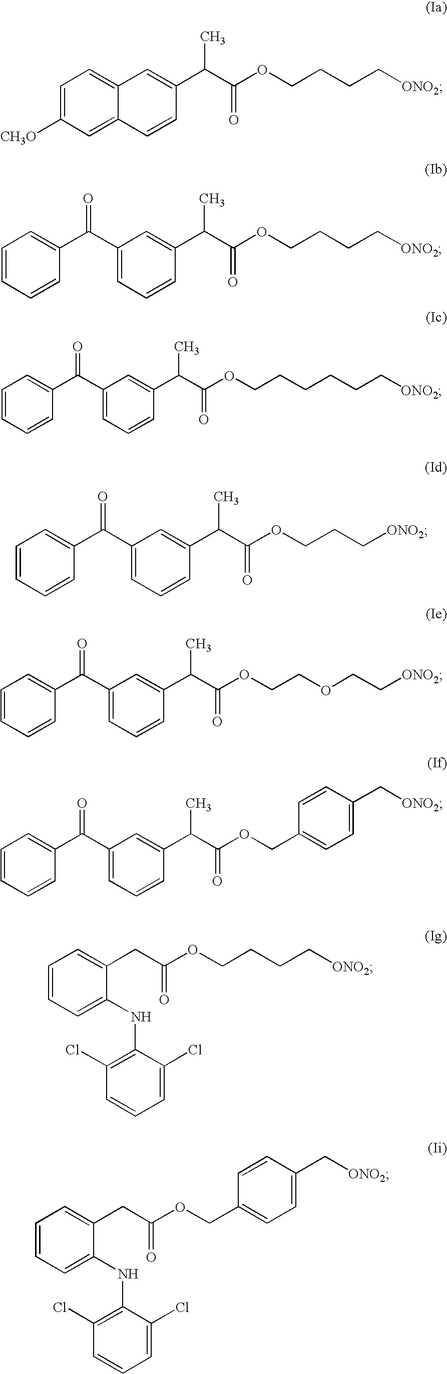

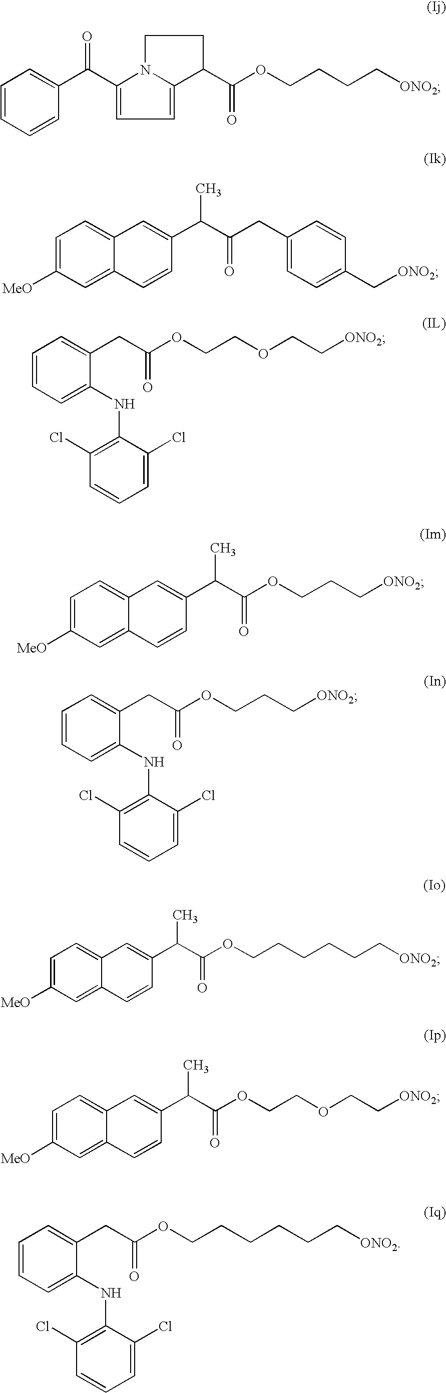

5. A pharmaceutical composition according to claim 4 wherein the NO-releasing NSAID is a compound according to formula Ia.

6. A pharmaceutical composition according to claim 4 wherein the NO-releasing NSAID is a compound according to formula Ic, If, Ig or IL.

7. A pharmaceutical composition according to any one of the preceeding claims wherein the surfactant is selected from phospholipids, e.g. naturally occurring phospholipids; synthetic or semisynthetic phospholipids; ethoxylated phospholipids; galactolipids and other glycolipids; bile acids and their salts; sterols and esters therof; ethoxylated sterols; fatty acids and their salts; mono- and diglyceride esters of fatty acids; fatty acid esters and alcohols; ethoxylated fatty acids, ethers and esters; ethoxylated castor oil; ethoxylated sorbitan esters; polypropylenepolyethylene block copolymers such as poloxamers and poloxamines; or mixtures of two or more of these surfactants.

8. A pharmaceutical composition according to claim 7 wherein the surfactant is one of a naturally occurring, synthetic or semi-synthetic phospholipid; a polypropylene polyethylene block copolymer; an ethoxylated sorbitan ester; or a mixture of two or more of these surfactants.

9. A pharmaceutical composition according to claim 7 wherein the surfactant is a naturally occurring phospholipid from soya in combination with a poloxamer.

10. A pharmaceutical formulation according to claim 7 wherein the surfactant is polysorbate 80.

11. A pharmaceutical composition according to any one of claims 1-10 wherein the NO-releasing NSAID lipophilic core further comprises one or more lipophilic water-immiscible solvent(s).

12. A pharmaceutical composition according to claim 11 wherein the lipophilic water-immiscible solvent is a vegetable oil; a fractionated oil; a marine oil; an ester of a medium or long-chain fatty acid; a chemically modified or manufactured material; or a mixture of two or more of the above water-immiscible solvents.

13. A pharmaceutical composition according to claim 11 wherein the lipophilic, water immiscible solvent is fractionated coconut oil.

14. A pharmaceutical composition according to any one of the preceeding claims wherein the aqueous phase contains water and one or more of buffering agents; salts; pH adjusting agents; tonicity modifiers; water miscible solvents; density modifiers; viscosity modifiers agents; preservatives; antioxidants; and taste modifiers.

15. A pharmaceutical composition according any one of the preceeding claims wherein the amount of NO-releasing NSAID or mixtures of NO-releasing NSAID and water-immiscible solvent is up to 30% by weight of the composition.

16. A pharmaceutical composition according to claim 15 wherein the amount of NO-releasing NSAID or mixtures of NO-releasing NSAID and water-immiscible solvent is 0.5-20% by weight of the composition.

17. A pharmacutical composition according to any one of the preceeding claims wherein the amount of surfactant is up to 20% by weight of the composition.

18. A pharmaceutical composition according to claim 17 wherein the amount of surfactant is 0.1-10% by weight of the composition.

19. A pharmaceutical composition according to any one of the preceeding. claims for oral, rectal, parenteral, nasal or topical administration to a human or an animal.

20. Use of a pharmaceutical composition according to claims 1-18 for use in therapy.

21. Use according to claim 20 in the treatment of pain.

22. Use according to claim 20 in the treatment of inflammation.

23. A method for the treatment of pain which method comprises treating a subject suffering from said condition with a pharmaceutical composition according to any one of claims 1-18.

24. A method for the treatment of inflammation which method comprises treating a subject suffering from said condition with a pharmaceutical composition according to any one of claims 1-18.

25. A process for the preparation of a composition according to any one of claims 1-20 wherein
i) one or more surfactant(s) is added to an aqueous phase whereupon one or more NO-NSAID(s) is dispersed in the aqueous phase by using conventional dispersion techniques such as high shear mixing, sonication or high pressure homogenisation; or
ii) one or more NO-NSAID(s) is mixed with one or more surfactant(s), whereupon the mixture is dispersed in an aqueous phase by using conventional dispersion techniques such as high shear mixing, sonication or high pressure homogenisation; or
iii) one or more surfactant(s) is added to an aqueous phase and one or more NO-NSAID(s) is mixed with one or more lipophilic water-immiscible solvent(s), whereupon the mixture of NO-NSAID(s) and lipophilic water-immiscible solvent(s) is dispersed in the aqueous phase by using conventional dispersion techniques such as high shear mixing, sonication or high pressure homogenisation; or
iv) one or more NO-NSAID(s) is mixed with one or more surfactant(s) and one or more lipophilic water-immiscible solvent(s), whereupon the mixture is dispersed in an aqueous phase by using conventional dispersion techniques such as high shear mixing, sonication or high pressure homogenisation; or
v) one or more surfactant(s) is added to the aqueous phase, and one or more surfactant(s) is mixed with one or more NO-NSAID(s), whereupon the mixture of NO-NSAID(s) and surfactant(s) is dispersed in the aqueous phase by using conventional dispersion techniques such as high shear mixing, sonication or high pressure homogenisation; or
vi) one or more surfactant(s) is added to the aqueous phase, and one or more surfactant(s) as well as one or more lipophilic water-immiscible solvent(s) is mixed with one or more NO-NSAID(s), whereupon the mixture of NO-NSAID(s), surfactant(s) and lipophilic water-immiscible solvent(s) is dispersed in the aqueous phase by using conventional dispersion techniques such as high shear mixing, sonication or high pressure homogenisation.

26. A process according to claim 25 wherein the NO-releasing NSAID(s) is heated above its melting point prior to dispersion in the aqueous phase.
